(19) United States
(12) Patent Application Publication
(10) Pub. No.: US 2018/0000999 A1
(43) Pub. Date: Jan. 4, 2018

(54) DEVICES AND METHODS FOR FLUID INFUSION, DRAINAGE, AND COLLECTION

(71) Applicant: Merit Medical Systems, Inc., South Jordan, UT (US)

(72) Inventors: Bart Dolmatch, Palo Alto, CA (US); Celso Bagaoisan, Union City, CA (US); Suresh Subraya Pai, Los Altos, CA (US); Fabio Komlos, Mountain View, CA (US)

(21) Appl. No.: 15/696,440

(22) Filed: Sep. 6, 2017

Related U.S. Application Data
(63) Continuation of application No. PCT/US2016/040174, filed on Jun. 29, 2016.

(51) Int. Cl. A61M 1/00; A61M 39/10; A61M 5/142; A61M 39/24

(52) U.S. Cl. CPC A61M 1/0011; A61M 1/0027; A61M 1/0035; A61M 1/0003; A61M 5/14224; A61M 39/10; A61M 1/0017; A61M 2205/0216; A61M 2205/3379; A61M 2205/583; A61M 2205/584; A61M 2205/587; A61M 2209/088; A61M 39/24

(57) ABSTRACT

Containers for collecting fluid are provided that include a reservoir comprising a plurality of panels enclosing an interior, side panels of the reservoir are movable from an expanded position towards to a compressed position and biased to the expanded position for generating a vacuum within the interior. An elongate housing extends along a top side of the reservoir including a passage extending between proximal and distal ends thereof and communicating with the interior of the reservoir. An elongate member extends through the housing including a distal extension, a proximal extension, and a lumen extending therebetween. One-way valves are coupled to the distal and proximal extensions for permitting fluid flow proximally from the distal extension into the interior of the reservoir via and permitting fluid flow proximally from the proximal extension while preventing fluid flow distally out the distal extension and distally into the proximal extension and lumen.

### RELATED APPLICATION DATA

This application is a continuation of International Patent Application No. PCT/US2016/040174, filed on Jun. 29, 2016, and published as WO 2017/007660, which claims the benefit of (1) U.S. Provisional Application No. 62/317,766, filed Apr. 4, 2016, and (2) U.S. Provisional Application No. 62/188,601, filed Jul. 3, 2015. Each of these applications is hereby incorporated by reference in its entirety.

### BACKGROUND

There are many reasons that external drainage of normal and abnormal body fluids or infusion or instillation of fluids or medications into the body must be performed. Some of these conditions include the need for drainage of pus, stool, urine, bile, serous fluid, lymph, gastric or enteric contents, or blood; the need for instillation or infusion of fluids or medications into organs such as the stomach or intestines, renal collecting systems, biliary tree, abscess cavities, seromas, lymphoceles, hematomas, bladder, chest cavity, or peritoneal cavity. Often, surgical and image-guided procedures leave an indwelling tube (drain or drainage, infusion, or instillation catheter) that passes from the internal bodily structures or collections through the skin to an external drainage, infusion, or instillation system. The main components of a typical external drainage, infusion, or instillation system (beyond the drainage, infusion, or instillation catheter) generally comprise tubing, a reservoir for the collection of the bodily fluid, and often a valve that permits control and restricts or permits access to the drainage, infusion, or instillation system. The tubing may comprise any sort of flexible conduit with at least one lumen along its length through which the bodily fluid passes. The collection reservoir may comprise a simple bag (e.g., that allows drainage, infusion or instillation by passively or via the assistance of gravity) or a suction system whereby bodily fluid is withdrawn from the body via the assistance or urging of negative pressure or a vacuum force.

Free flow of bodily fluid through the system must be assured while a typical drainage, infusion, or instillation catheter is in place. As many types of internal body fluids and collections often contain debris, clot, mucous, coagulated proteins, stones, or other substances that can obstruct the system, it is not uncommon that said substances sometimes inhibit or prevent unimpeded drainage, infusion, or instillation of bodily fluid. Free flow of material into or out of the body can also be reduced or obstructed by the viscosity of the materials itself or due to interference or less than ideal performance from components of the system. This can render the drainage, infusion, or instillation system less functional or sometimes useless, and therefore the underlying problem may not be treated as clinically indicated or desired by the physician. When this occurs, the dwell time or residence of such a drainage, infusion, or instillation catheter in the patient's body may be prolonged and patient safety and comfort will be compromised.

Regarding drainage of internal body fluid collections, different mechanisms may be in play. Sometimes the mechanism of drainage is passive, while other times, the mechanism is by means of gravity, and still other times via a suction system. Passive drainage relies upon the build up of pressure within the fluid collection inside the body, such that merely by inserting a tube into the fluid there is adequate pressure within the fluid for it to drain externally. For example, if there is continuous excretion of urine, then over time the urine will pass into the urinary bladder and distend the bladder wall. The muscular tone of the distended bladder wall causes more pressure within the bladder than outside the body. In this case, passive drainage will occur when a catheter placed into the bladder simply because there is more pressure within the bladder than outside of it.

With gravity drainage, fluid will flow downhill to a dependent collection bag or reservoir. An example of gravity drainage is insertion of a tube into a fluid collection that is not under pressure, such as a partially decompressed seroma. Here, drainage occurs when the collection reservoir is lower than the level of the seroma because the fluid will flow “downhill” via gravitational force.

The third mechanism of drainage (i.e., a suction system) produces lower pressure (suction or vacuum) when compared to the pressure of the fluid collection within the body. Suction systems include drainage bulbs like the “Jackson Pratt” bulb well known in the art. The bulb is first squeezed and deformed to remove the volume of air within it. Subsequently, since the bulb is constructed from elastic material (e.g., typically Silicone rubber), it seeks to assume its non-collapsed state by expanding to return to its pre-set shape. There are also various other reservoirs that have spring-type mechanisms to force the expansion of the collapsed device back to its resting state. This type of drainage system is very commonly used for post-operative fluid drainage, abscess drainage, and drainage of hematomas, infected hematomas, sterile and infected pancreatic fluid, and for active drainage of seromas and other types of fluid collections when aggressive fluid removal is clinically required or desired.

Most drainage depots, regardless of mechanism of action, are poorly designed from the perspective of patient wearability. While many can be affixed to the body using safety pins, adhesive straps, or belts, the reservoirs used today are typically ill-fitting, aesthetically unappealing and awkward to handle. For example, the Jackson Pratt bulb is cumbersome since it doesn't fit easily into a pocket and therefore it is usually worn as an external bulbous translucent device attached to clothing by either a safety pin or by means of a loop of plastic through which a belt can be passed. Whether pinned or suspended from a belt, the bulb is often an obvious and aesthetically unappealing appliance that cannot be easily concealed. Other reservoir systems are designed to be strapped to the leg (e.g., small uninary and biliary drainage bags) and some cannot be easily attached to the body at all (e.g., large urinary drainage bags and enteral solution reservoirs). Therefore, there exists a need for a wearable, compact, unobtrusive fluid collection reservoir.

Systems that are used for infusion or instillation, drainage, or collection of fluids often permit easy visualization of contents within. When there is drainage of fluid, the contents may be purulent, feculent, bilious, bloody, and the fluid may contain debris, clots, mucous, urine, and/or other body wastes. These contents are often unsightly, yet poorly concealed within the reservoir. Enteral nutrition may be infused from a reservoir, and is typically somewhat viscous and opaque, and also not at all appealing. Various therapeutic fluids of many different colors and viscosities may be required for infusion or instillation, and today's fluid collection reservoirs do not conceal the appearance of these fluids, thereby allowing others to deduce the contents and perhaps the condition for which the patient is being treated. Visualization of the contents of a fluid reservoir compromises the patient's ability to maintain privacy.

Beyond issues of a discrete and wearable fluid reservoir, the reservoirs commonly used today are prone to malfunction, soilage, and transmission of contaminated fluids. For example, if the stopper on a Jackson Pratt bulb pops off (it is only held in place by friction), then the vacuum is lost and fluid within the bulb can inadvertently drain onto the patient, clothing, furniture, floor, and others in the immediate vicinity. Infectious agents such as bacteria, fungi, and viruses may be communicable and can be spread to others. Furthermore, loss of vacuum, if it occurs, may render this sort of drainage system less effective since drainage would only occur by passive or gravity mechanisms. If the valve on a passive fluid collection bag is unknowingly twisted open, then similar soilage may occur.

While malfunction of a vacuum system may seem hypothetical, it occurs regularly. During sleeping hours, the patient may roll onto the system, dislodging the stopper or torquing the drainage valve. Lying upon the reservoir can also pressurize the fluid within, causing it to leak out of the drainage port of the fluid collection reservoir. If there is loss of vacuum at night, it may persist for many hours until recognized and may possibly create morbidity issues. Bulky or bulbous drainage systems can also interfere with the patient's ability to sleep due to the size and position of the reservoir.

Mechanisms that alert the patient to loss of suction do not exist in present systems. For example, if a suction bulb is nearly full the wearer is at a loss to know if there is any residual vacuum. Other spring mechanism suction reservoirs are likewise devoid of a gauge or indicator that shows the persistence or loss of vacuum as they become full or should they lose vacuum for any other reason.

Finally, while ongoing collection of body fluids, instillation or infusion of fluid is certainly not an enjoyable experience for most patients, the reservoirs that are used today are not discrete or unobtrusive. These bulky and unsightly fluid depots can impede return to a normalized lifestyle for most patients who may otherwise able to return to their homes and often to their jobs. It is common for friends, family, and coworkers to notice the patient's infirmity. Social and psychological stress may be associated with most of the reservoirs used today. Perhaps of greater concern is the psychological distress when spillage occurs, be it in the home or when the patient is in a public place.

### SUMMARY

Thus, with these notable shortcomings, it is apparent there is a need for improved devices and methods for the application of vacuum or negative pressure to enable the drainage, infusion, and instillation of fluids in a body. Exemplary embodiments provided herein may include, but are not limited to, one or more of the following features:
a. Devices that have a generally low profile and smooth contours/surfaces and interfaces that are specifically designed with the intent to mitigate the potential for pain and wound creation if the device is pressed against the skin, as well as to facilitate usage and overall wearability underneath clothing without being too cumbersome or noticeable.
b. Devices that include features that make them foolproof with the intent of preventing inadvertent and/or accidental drainage of contaminated fluids onto the patient or into the environment about the patient.
c. Devices that include features that prevent or mitigate inadvertent and/or accidental flow of contaminated fluids into the body.
d. Devices that include features that prevent or mitigate inadvertent and/or accidental loss of vacuum pressure to the system.
e. Devices that include lumens and ports having dimensions that specifically optimize and maximize flow of bodily fluids and medications while minimizing the opportunity to become obstructed by debris.
f. Devices that comprise features for presenting information about the internal pressure condition of the device to the patient or physician or caregiver.
g. Devices that comprise features for presenting information about the volume of effluent collected and/or contained within the device.
h. Devices that comprise features to easily switch between the different functional states of a drainage/infusion/instillation system.
i. Devices that have a general aesthetic appeal intended to reduce patient's embarrassment or anxiety related to such devices, facilitate usage and reduce the encumbrance to the patient's day-to-day life and routines.
j. Methods of use for the aforementioned devices.

Described herein are devices that provide negative pressure or vacuum as a motive force enabling the drainage of fluid from a body lumen, cavity, anatomical structure, or the like along with methods of using the same. The devices generally comprise a housing, at least one elongate member resident within and extending beyond the edges of the housing, and one or more features for generating negative pressure connected to the housing.

In one embodiment, the device generally comprises a housing, at least one elongate member, a flexible enclosure, and a mechanism for stretching or expanding the flexible enclosure. The housing may be fabricated from materials known in the art including, but not limited to, aliphatic polyamides, fluorinated ethylene propylene, nylon, perfluoroalkoxy (e.g. Teflon®), polyether block amide (Pebax®), polyetheretherketone (PEEK), polyethylene, polytetrafluoroethylene (PTFE), polypropylene, polyurethane, polyvinylchloride, polysulfone, stainless steel, nickel, titanium, aluminum, brass, copper, polycarbonate, acrylic, polyoxymethylene (Delrin®), combinations and/or alloys thereof, and the like. The housing may be fabricated from a monolithic block of material using methods known in the art including but not limited to injection molding, manual or computer numerically controlled machining, combinations thereof, and the like, or alternatively, may be fabricated via the assembly of numerous subcomponents using methods known to the art including, but not limited to, threading/tapping, bonding, ultrasonic welding, press or interference fitting, snaps, pins, combinations thereof, and the like.

The housing may additionally comprise openings, gaps, or spaces in the body of the housing. The openings, gaps, or spaces may be shaped to enable a variety of additional components to be mounted on, slidably disposed within, or otherwise interact with the housing. In one example, the housing may comprise at least one lumen extending throughout the length of the housing. The shape and dimensions of the at least one lumen may be chosen to allow free passage of one or more elongate members through the body of the housing. Alternatively, the shape and dimensions of the at least one lumen may be selected to interfere with the outer surfaces of the one or more elongate members to, for example, fix the relative positions of the elongate member and the housing. The housing may additionally comprise seals, gaskets, compression features, and the like to provide an air or water-tight seal between the elongate member and the housing. The housing may additionally comprise an opening, gap, or space that allows the at least one lumen to communicate with other spaces or locations within the device.

The housing may further comprise an opening or cavity sized to accept a complementary component of the device. For example, a key may be stored on the housing in an appropriately sized pocket in a face of the housing. In some cases, an opening may be positioned such that visual inspection of an internal feature of the device is possible. For example, an opening may be aligned with the length of the housing to enable the patient to confirm the volume of exudate present in the device. This opening may further be covered by a translucent or transparent material to enable view of the exudate while preventing physical access to the interior of the device (e.g., to act as a sight glass).

The translucent or transparent materials may be joined to the housing using methods known to the art including, but not limited to, bonding, threading/tapping, screws, ultrasonic welding, interference fits, press fits, pins, tacks, overmolding, combinations thereof, and the like. The translucent or transparent material may be fabricated from materials known to the art including, but not limited to, polycarbonate, polymethylmethacrylate, glycol modified polyethylene terephthalate, polyvinylchloride, cyclic polyolefins, polystyrene, nylon, acrylonitrile butadiene styrene, glass, and the like.

The translucent or transparent material may further comprise gradations to assist in determining the volume or amount of exudate collected in the device. The gradations may be formed using methods known to the art including, but not limited to, pad printing, inscription, molding, etching, combinations thereof, and the like. In another example, the gradations may be located on the housing adjacent to the transparent or translucent material. It should be clear to one skilled in the art that a feature such as a translucent or transparent window may be expanded to locations beyond those explicitly stated, and that alternative uses and positions of this feature have been duly contemplated.

In another example, a housing may comprise one lumen extending from the proximal to distal end of the housing, and an external recess or cavity extending along the length of the housing from the distal end of the housing to a point distal to the proximal end of the housing. The lumen and recess may be oriented parallel to each other. The first lumen may be sized to accept an elastic, tubular elongate member of a given outer diameter. While the recess or cavity may be of any size or shape, the recess or cavity may be circular in cross section and positioned such that a chord of the cross sectional plane of the recess is exposed to the external environment. The second lumen may be sized to securely hold the elongate member. For example, the diameter of the recess may be less than that of the elastic elongate member. Pushing the elongate member into the recess compresses the elastic elongate member radially and provides an outward force against the internal surfaces of the recess, securing the elongate member in the recess.

The recess may further comprise a segment that is sized to accept a component located on or along the elongate member. The component may be any of a number of fittings known to the art including, but not limited to, valves, male or female luer-locks (fixed or rotating), male or female luer-slips, quick-disconnect fittings, hose barbs, internally threaded fittings, externally threaded fittings, combinations thereof, and the like. For example, the elongate member may comprise a duckbill valve and male luer-lock fitting positioned at the distal end of the elongate member. The expanded segment of the recess may be located at any location along the length of the recess, for example, positioned at the proximal end of the recess. The expanded segment allows the elongate member to be secured in a position substantially inside the housing in the following manner:
a. The male luer-lock fitting at the distal end of the elongate member is grasped and pulled perpendicular to a longitudinal axis of the housing and away from the side of the housing that contains the housing lumen (e.g., if the housing lumen is on the right-hand side of the housing in a top-down view, the elongate member is pulled to the left of the housing until the elongate member is perpendicular to the long axis of the housing).
b. The elongate member is drawn towards the proximal end of the housing while maintaining a degree of tension on the elongate member.
c. The duckbill valve and male luer-lock fitting are placed inside the expanded segment of the recess wherein the tension on the elongate member pulls the larger components against the stop formed by the smaller diameter of the recess.
d. The remainder of the distal portion of the elongate member is pressed into the recess.

The “doubling back” of the elongate member as it is pulled through an approximately 180° turn provides a method for closing off the lumen of the elongate member and preventing flow through portion of the elongate member distal to the distal end of the housing. This feature provides the patient with a method to store the distal portion of the elongate member when it is not in use, and allows for a smooth and compact profile for the device.

The housing may be shaped and/or contoured to fit an anatomical structure. For example, a housing intended for mobile, personal use may possess a curved shape that fits comfortably along the waist of a patient. The width of the housing may be relatively narrow with respect to the length of the housing, providing a slim profile that does not catch on clothing or other items while the patient is active. For example, the depth of the housing may be less than or equal to one third of the length of the housing. The external surfaces of the housing may further comprise curved and/or flowing contours such that sharp angles and/or protrusions that would produce discomfort if laid upon when a user of said apparatus is sleeping are limited or non-existent. Additionally, the external surface of the housing may comprise features such as rounded or tapered edges, textured segments wherein the surface of the housing is rougher or smoother than surrounding areas, patterns of or combinations thereof, and the like to further improve patient comfort and ease of use. In side profile, the device as a whole may be generally square or generally rectangular in shape.

The housing may further comprise features that enable the housing to be carried by, on, or mounted to the patient. In one example, an opening may be configured to allow a lanyard, tie, cord, cable, rope, string, belt, or other means of securing the device to an external object (e.g., the patient) to be passed through the device. In another example, the housing may comprise a loop that projects away from the body of the device, wherein the loop is sized to allow passage of a cord through the loop. In yet another example, the housing may comprise a tubular opening with proximal and distal ends. The proximal and distal ends may be located on the same or disparate faces of the housing to form an enclosed conduit sized to accept a feature for securing the device to an external object.

In still another example, at least one face of the housing may comprise a loop or set of loops that are sized to enable the device to be carried on a belt, band, webbing, suspender, or other like object. Alternatively, at least one face of the housing may comprise a “U” shaped feature that extends away from the housing to enable the device to be hung on a belt, waistband, or like object. Furthermore, the housing may comprise a segment of adhesive that allows the device to be directly mounted onto the patients skin.

In an additional example, a holster or pocket may be fabricated from materials that comprise any or all of the following characteristics: lightweight, breathable, water resistant, elastic, waterproof, durable. The holster or pocket may further comprise one or more features to secure the device within the holster or pocket while providing access to the complementary parts of the drainage or infusion/instillation system (e.g., infusion lines, drainage or infusion/instillation catheter lines, etc.). The device may be secured by a flap that encloses the device and is reversibly fixed in place using features known in the art including, but not limited to, ties, hook-and-loop fasteners (e.g., Velcro®), buttons, snaps, grommets, zippers, clips, buckles, combinations thereof, and the like. The holster or pocket may have opening, holes, or other passageways that allow the device to reside inside the holster or pocket while connected to the complementary parts of the drainage or infusion/instillation system.

The holster or pocket may further comprise one or more features to attach the holster directly to the patient, to the clothes of the patient, or to other items. For example, a lightweight, elastic material (e.g., spandex) may be fabricated into a pocket that securely holds the device and comprises multiple openings for the complementary parts of the drainage or infusion/instillation system. The holster pocket may also have a double-sided adhesive suitable for adhering the pocket to the skin of a patient affixed to one or more sides of the pocket, enabling the patient to wear the device underneath clothing. Alternatively, the holster or pocket may comprise at least one grommet and at least one lanyard (wherein the lanyard is passed through the grommet) to allow the pocket to be worn on the arm, about the neck, or the like.

In another example, the holster may additionally comprise at least one length of webbing that is reversibly or permanently fixed to the holster, wherein the ends of the webbing comprise complementary halves of a hook-and-loop fastener. The at least one length of webbing may be passed around the arm, leg, waist, or the like of the patient and reversibly fixed in position by mating the two halves of the hook-and-loop fastener. In yet another example, the webbing of the prior example may be elastic.

The at least one elongate member may be fabricated from materials known to the art including, but not limited to, aliphatic polyamides, fluorinated ethylene propylene, nylon, perfluoroalkoxy (e.g., Teflon®), polyether block amide (Pebax®), polyetheretherketone (PEEK), polyethylene, polytetrafluoroethylene (PTFE), polypropylene, polyurethane, polyvinylchloride, natural rubber, nitrile rubber, silicone rubber, combinations and copolymers thereof, and the like. The elongate member may further comprise at least one lumen extending through the length of the housing. The inner diameter of the at least one lumen of the elongate member may be sized to accommodate the exudate, body fluid, or other material that the device is intended to drain. The elongate member or members may have identical or dissimilar numbers of lumens, and the inner diameter of the lumens may be identical, dissimilar, or combinations thereof. Similarly, the outer diameters and lengths of the elongate member or members may be identical, dissimilar, or combinations thereof. Further, the wall and/or the outer diameter of the elongate member or members may be constant or variable in dimension. The elongate member or members may further comprise a fixed or variable stiffness over the length of each elongate member, and a particular stiffness or variation in stiffness along the length of the elongate members may be identical or dissimilar, or a combination thereof.

The elongate member may be disposed within an opening of the housing and of a length that allows the proximal and distal ends of elongate member to extend past the proximal and distal ends of the housing. The elongate member may be joined to the aforementioned opening of the housing using methods known to the art including, but not limited to, bonding, welding, ultrasonic welding, over-molding, friction or interference fits, combinations thereof, and the like. The elongate member may further comprise an opening in the wall of the elongate member enabling communication between a lumen of the elongate member and a second space in the housing or in the device. The opening may be sized and oriented to optimize flow of exudate from the lumen of the elongate member, through the housing, and to, for example, a collection bag, bellows, vacuum source, or the like incorporated in the device.

The proximal and/or distal ends of the elongate member may be joined to other components including, but not limited to, valves, male or female luer-locks (fixed or rotating), male or female luer-slips, quick-disconnect fittings, hose barbs, internally threaded fittings, externally threaded fittings, flexible tubing, and the like. The other components may be fabricated from materials known to the art including, but not limited to, polycarbonate, polyethylene, polyolefin, polypropylene, polytetrafluoroethylene, polysulfone, polyvinylchloride, polyoxymethylene (Delrin®), brass, stainless steel, nylon, perfluoroalkoxy (e.g., Teflon®), natural rubber, nitrile rubber, silicone rubber, combinations thereof, and the like. The distal and proximal ends of the elongate member may be joined to the other components using methods known to the art including, but not limited to, bonding, welding, ultrasonic welding, over-molding, threading/tapping, crimping, combinations thereof, and the like. For example, the proximal end of the elongate member may be joined to a one-way valve oriented such that flow is permitted in the proximal to distal direction and prevented in the distal to proximal direction. The one-way valve may further comprise a female luer-lock on the proximal end of the valve to facilitate connection of the device of the invention to other medical devices (e.g., an infusion catheter, stopcock, etc.).

In another example, the distal end of the elongate member may be joined to a one-way valve oriented such that flow is permitted in the proximal to distal direction and prevented in the distal to proximal direction. The one-way valve may further comprise a male luer-lock on the proximal end of the valve to facilitate connection of the device of the invention to other medical devices (e.g. a surgical drain, etc.). While these examples specify male and female luer-locks as the type of connector, it should be clear to one of skill in the art that a myriad of connecting mechanisms may be used to reversibly or irreversibly join the device of the invention to other medical devices or equipment.

In an alternative embodiment, the housing of the device comprises features such as ports that enable elongate members to reversibly or irreversibly join to the housing at the proximal and distal ends of a lumen within the housing. A housing intended to reversibly join to one or more elongate members may comprise features including, but not limited to, male or female luer-locks (fixed or rotating), male or female luer-slips, quick-disconnect fittings, hose barbs, internally threaded fittings, externally threaded fittings, and the like positioned at the proximal and distal ends of the lumen of the housing. An elongate member comprising a complimentary feature would then be able to reversibly join to the housing and provide flow path between the lumen of the elongate member and the lumen of the housing.

For example, an elongate member comprising a male luer-lock located at one of its ends could reversibly join a housing comprising a female luer-lock positioned at one end of the housing lumen. The specific features for coupling the elongate members to the housing may be similar or dissimilar (e.g., male luer-locks at the proximal and distal ends of the housing lumens, or a male luer-lock at the proximal end of the housing lumen and a hose barb at the distal end of the housing lumen).

It should be clear to one of skill in the art that a wide variety of features, couplings, and connectors may be employed to reversibly join the housing to one or more elongate members. Furthermore, the aforementioned features, couplings, and connecters may serve to reversibly join the housing of the device to other medical devices or equipment. A housing intended to irreversibly join to one or more elongate members may comprise features including, but not limited to, flanges, extruded rings, undercuts, countersinks, barbs, combinations thereof, and the like. An elongate member may be joined to these features using methods known to the art including, but not limited to, bonding, welding, ultrasonic welding, over-molding, threading/tapping, crimping, press fitting, interference fitting, combinations thereof, and the like.

For example, a housing comprising two complementary halves and a lumen may further comprise two expanded portions of the lumen that are larger in size than the majority of the lumen. For example, a first expanded portion may be located a distance offset away from the proximal end of the housing and a second expanded portion may be located a distance offset away from the distal end of the housing. An elongate member comprising a flange of a size and shape to fit in the expanded portion may be joined to either the distal or proximal side of the housing by inserting the flange into the corresponding expanded portion and fixing the two complementary halves of the housing to one another. The elongate member would be fixed in position relative to the housing and provide a continuous open path from the lumen of the elongate member to the lumen of the housing. This process may be repeated with a second elongate member and the second expanded portion prior to fixing the halves of the housing to one another, such that a continuous flow path exists from the lumen of the proximal elongate member, through the lumen of the housing, and to the lumen of the distal elongate member. It should be clear to one of skill in the art that the joints as described herein would be complemented with the appropriately designed gaskets, o-rings, seals, and the like to support adequate internal positive or negative pressures within the device.

In an exemplary embodiment, the mechanism for generating negative pressure may comprise two plates connected by one or more torsion springs and a flexible membrane surrounding the plates and torsion springs and affixed to the housing. The two plates and one or more torsion springs may be fabricated from materials known in the art including, but not limited to, aliphatic polyamides, fluorinated ethylene propylene, nylon, perfluoroalkoxy (e.g., Teflon®), polyether block amide (Pebax®), polyetheretherketone (PEEK), polyethylene, polytetrafluoroethylene (PTFE), polypropylene, polyurethane, polyvinylchloride, polysulfone, stainless steel, nickel, titanium, aluminum, brass, copper, polycarbonate, acrylic, polyoxymethylene (Delrin®), combinations and/or alloys thereof, and the like.

The torsion springs may be joined to the plates using methods known to the art including, but not limited to, bonding, welding, ultrasonic welding, over-molding, threading/tapping, crimping, press fitting, interference fitting, combinations thereof, and the like. The plates may be any size or shape, for example, substantially as long as the housing. The edges of the plates may be filleted or chamfered to minimize the risk of cutting or perforating the flexible membrane.

The flexible membrane may be fabricated from materials known in the art including, but not limited to, aliphatic polyamides, fluorinated ethylene propylene, nylon, perfluoroalkoxy (e.g., Teflon®), polyether block amide (Pebax®), polyetheretherketone (PEEK), polyethylene (e.g. Tyvek®), polytetrafluoroethylene (PTFE), polypropylene, polyurethane, polyvinylchloride, natural rubber, nitrite rubber, silicone rubber, combinations and copolymers thereof, and the like. The flexible membrane may be transparent, translucent, or opaque and the surfaces may be smooth, textured, or a combination thereof. Furthermore, the flexible membrane may be impermeable or semipermeable to materials including, but not limited to, gases (e.g., water vapor), liquids (e.g., water), proteins or molecules of a given size or range of sizes, combinations thereof, and the like.

The assembly of the plates and one or more torsion springs is such that when the one or more springs are in the unloaded state, the plates are positioned such that the planes formed by the plates are at an angle to each other. For example, the internal edges of the plates may be connected to each other via the one or more torsion springs. In an unloaded state the one or more torsion springs hold the plates at a 45° angle from each other. When pressure is applied to the outer surface of the plates, the one or more torsion springs are compressed and the plates advance towards each other. Under sufficient pressure the plates will contact each other. The torsion springs will return the plates to the original 45° orientation when pressure is removed from the outer surfaces of the plates. While an angle of 45° is described herein, it should be clear to one of skill in the art that any angle between 0° and 360° between the plates is contemplated.

The assembly of the plates and one or more torsion springs may be joined to the housing using methods known to the art including, but not limited to, bonding, welding, ultrasonic welding, over-molding, threading/tapping, crimping, press fitting, interference fitting, combinations thereof, and the like. The flexible, impermeable membrane may be shaped to fit around the exterior of the plates and attach to the housing such that an air-tight seal is formed between the membrane and the housing, and the assembly of plates and one or more torsion springs is enclosed within the membrane. The membrane may be joined to the housing using methods known to the art including, but not limited to, bonding, welding, ultrasonic welding, over-molding, threading/tapping, crimping, press fitting, interference fitting, combinations thereof, and the like.

In an alternative embodiment, the features for generating vacuum may be an extension of the housing. For example, the two plates of the previous example may extend from the base of the housing in a generally parallel orientation and be of a stiffness and dimension that they return to their original position when deformed. The plates may be surrounded by or embedded within a flexible impermeable membrane as previously described. The plates will advance towards each other under the application of force to the outer surfaces of the plates. Under sufficient force the plates may contact each other. The inherent stiffness and dimension of the plates will return the plates to the original generally parallel orientation when force applied to the outer surfaces of the plates is reduced or released.

This example for generating vacuum is not restricted to a generally parallel orientation of the two plates. On the contrary, any orientation of the plates relative to each other is contemplated. For example, an angle between the plates is contemplated. In such an embodiment, the plates may originate at a distance apart from each other on the housing with the terminal edges of the plates a greater distance apart from each other than at the origin. A side view of such an arrangement may generally be trapezoidal in shape, with the larger of the two parallel sides furthest away from the housing.

In yet another example, the cross section of the features for generating vacuum may have the shape of an isosceles trapezoid with the larger of the two parallel sides furthest away form the housing. Furthermore, the volumes of space bounded by the outer surface of the plates and projections extending downward from the external faces of the housing may be filled with material to create a generally rectangular form to the device (as opposed to a generally trapezoidal shape suspended below a generally rectangular shape).

In this example, the housing further comprises a flow path to the external environment that can be opened or closed. Opening the flow path and compressing the plates decreases the volume of space contained within the membrane. Upon closing the flow path and releasing pressure on the plates, the plates will attempt to return to their resting orientation, increasing the volume of space within the membrane and generating a negative pressure within the device. Opening the flow path to the external environment will draw air or fluid into the volume of space contained within the membrane. Alternatively, the two plates may be fabricated as a living hinge as a mechanism for generating negative pressure within the flexible membrane. The mechanism of action is identical to that of the plate and torsion spring assembly. In another example, the plates may be partially, substantially, or fully embedded within the flexible membrane such that flexing or otherwise moving the plates directly stretches or compresses the impermeable membrane.

Optionally, the device may additionally comprise one or more features for presenting information about the pressure within the device to the patient or user. For example, the housing may comprise a cavity that is in fluid communication with the volume of space within the flexible membrane. The cavity may be located on the external surface of the housing in a location readily observable by the patient or user. The open surface of the cavity may be sealed by a vacuum pressure indicator comprising a flexible, impermeable membrane fabricated from materials known to the art including, but not limited to, aliphatic polyamides, fluorinated ethylene propylene, nylon, perfluoroalkoxy (e.g., Teflon®), polyether block amide (Pebax®), polyetheretherketone (PEEK), polyethylene, polytetrafluoroethylene (PTFE), polypropylene, polyurethane, polyvinylchloride, natural rubber, nitrile rubber, silicone rubber, combinations and copolymers thereof, and the like.

The vacuum pressure indicator may be shaped to fit the cavity and joined to the housing such that an air-tight seal is formed between the membrane and the housing using methods known to the art including, but not limited to, bonding, welding, ultrasonic welding, over-molding, threading/tapping, crimping, press fitting, interference fitting, combinations thereof, and the like. The vacuum pressure indicator may be shaped such that under neutral or positive internal pressure the vacuum pressure indicator protrudes from the surface of the housing. For example, the flexible, impermeable membrane of the vacuum indicator may be domed or hemispherical in shape under positive pressure, wherein the curved surface of the dome extends away from the body of the housing. Under negative pressure the flexible membrane is pulled partially, substantially, or fully into the cavity. The user can use the visual or tactile information presented by this exemplary embodiment of the vacuum pressure indicator to determine the binary state of the pressure within the device of the invention (i.e., positive or negative pressure).

Alternatively, the vacuum indicator may provide more detailed information such as the magnitude of positive or negative (i.e., vacuum) pressure within the device. For example, the housing may comprise a gage in fluid communication with the volume of space within the flexible membrane that comprises an assembly that displays a numerical value representative of the pressure within the device of the invention. The gage may be fully mechanical, such as in a tire pressure gage well known in the art, or the gage may further comprise electrical components to provide visual, audible, or tactile feedback such as a power source, transducer, light emitting diode (LED), a sound buzzer, or a vibration element such as those used in traditional, modern day cellular phones, any combination thereof, and the like.

For example, a gage may be configured to activate a single LED to provide the user with a visual cue or feedback when the pressure inside the device is within, above or below a pre-specified value. Alternatively, the gage may be configured with a series of LEDs or seven-segment displays to present more detailed information about the pressure within the device. For example, a series of individual LEDs may be aligned with markings on the surface of the housing that describe general information about the pressure within the device, such as “good,” “adequate,” “high,” or “low.”

Alternatively, the series of individual LEDs of different colors, or a single RGB LED may be aligned with a single cavity or port on the housing to display a color that communicates information about the level of pressure within the device of the invention. For example, the display of a green color may indicate and signify adequate vacuum pressure within the device, the display of an yellow color may indicate a low level of vacuum, and the display of a red color may indicate ambient or greater pressure.

A series of seven-segment displays may alternatively used to convey this information to the user. It is conceived herein that such information may be also shared wirelessly to other devices such as smart phones, computers and tablets. This information would be also useful to the user, physician, or caregiver to ensure the correct state of the device (i.e., the presence of vacuum pressure) and/or to provide a measure of visibility on the users compliance (i.e., user is utilizing the device correctly during the treatment window). The gage may optionally provide information as specific as the actual pressure within the device to any degree of accuracy and/or precision. Similarly, such information may be transmitted wirelessly and used with other electronic devices.

The device may be used to provide negative pressure to a drainage catheter, collect exudate from an abscess, provide an easy indication of the amount of exudate collected over a given span of time, and easily dispose of the exudate. In this exemplary method of use, the device comprises:
a. a generally rectangular housing further comprising a through lumen sized to accept and provide and air-tight seal about an elongate member and a port extending from the through lumen to the bottom face of the housing in a generally perpendicular manner. The size of the port is such that exudate (and any solid debris contained within the exudate) can easily flow through the port. The housing further comprises an opening covered with transparent material and gradations indicating the amount of exudate contained within the device.
b. an elongate member comprising a single through lumen, an opening sized to match the port of the housing, a duckbill valve oriented to allow flow from the distal to proximal directions located towards the distal end of the elongate member, a duckbill valve oriented to allow flow from the distal to proximal directions located towards the proximal end of the elongate member, a male luer fitting located at the distal end of the elongate member, and a female luer fitting located at the proximal end of the elongate member. The elongate member is disposed within the lumen of the housing such that the opening in the elongate member is aligned with the port of the housing.
c. a mechanism for generating negative pressure further comprising a living hinge arrangement of two plates attached to the bottom face of the housing, with the plates extending away from the bottom face of the housing at an approximately 30° angle from a vector normal to the bottom face of the housing (i.e., the distance between the plates increases as the distance between the point of measurement and the bottom face of the housing increases) embedded within a flexible opaque membrane, impermeable to gas and fluid, that surrounds the plates, defines a volume of space for collection of exudate, and connects in an air-tight manner to the housing. The port of the housing enables flow of exudate under negative pressure from the elongate member into the collection reservoir.

In use, the device is initially disconnected from the drainage catheter. The plates of the living hinge assembly are compressed and the air resident within the collection reservoir is expressed out of the collection reservoir, through the elongate member, and out of the duckbill valve located towards the proximal end of the elongate member. Compression of the plates is maintained while the male luer-lock located at the distal end of the elongate member is connected to the drainage catheter. The plates are released when a secure, air-tight connection between the male luer-lock and the drainage catheter has been achieved, creating a negative pressure within the device and applying that negative pressure to the drainage catheter (and subsequently to the abscess the distal end of the catheter is resident in). The air-tight seal between the elongate member and the housing along with the duckbill valve located at the proximal end of the elongate member prevent loss of negative pressure to the external environment.

Exudate is drawn from the drainage catheter and passes through the elongate member, opening in the elongate member, and the port in the housing before being deposited in the collection reservoir defined by the flexible membrane. The process of drawing exudate may continue until the pressure within the device equilibrates with ambient pressure or until a prescribed length of time has passed. To observe the amount of exudate collected, the drainage catheter is disconnected from the device and the housing is turned 90° such that the lowest gradation on the sight glass is oriented towards the bottom of the housing. The patient observes how far the exudate collected extends upwards through the sight glass using the gradations to note the volume collected.

To drain the collection reservoir of exudate, the plates are compressed to provide a positive pressure within the collection reservoir that exceeds the cracking pressure of the duckbill valve located at the proximal end of the elongate member. Exudate is pushed from the collection reservoir, through the housing port, through the proximal portion of the elongate member, through the duckbill valve located at the proximal end of the elongate member, and out of the elongate member. The duckbill valve located at the distal end of the elongate member and the air-tight seal between the elongate member and the housing prevent exudate from flowing out of the distal end of the device or leaking between the elongate member and the lumen of the housing.

Optionally, the device may further comprise a sheath that covers the housing and flexible membrane. The sheath may be fabricated from materials known to the art including, but not limited to, aliphatic polyamides, polyester, fluorinated ethylene propylene, nylon, perfluoroalkoxy (e.g., Teflon®), polyether block amide (Pebax®), polyetheretherketone (PEEK), polyethylene, polytetrafluoroethylene (PTFE), polypropylene, polyurethane, polyvinylchloride, polychloroprene, natural rubber, nitrile rubber, silicone rubber, cotton, flax, silk, wool, denim, canvas, linen, combinations and copolymers thereof, and the like. The sheath may be elastic or inelastic and may be knitted, woven, or non-woven in assembly. The sheath may be sized to fit about the housing and flexible membrane to provide a means of deflecting attention from the device. The sheath may also provide a method for carrying the device by passing a belt between the sheath and the flexible membrane, enabling the device to be carried by the patient in an inconspicuous manner.

The device may further comprise a valve device disclosed in U.S. provisional patent application 60/083,142, the entire disclosure of which is expressly incorporated by reference herein. The features of the valve device may be incorporated into the housing of the device in part or in full. The actuator valves of the valve device may be located at positions on the housing that are ergonomically convenient to the patient or user, and may optionally be shielded from inadvertent activation. The shielding may be a result of the design of the actuator valves as described in U.S. patent application 60/083,142, or comprise at least one physical interference lock or safety, at least one removable cover, or the like. It should be clear to one of ordinary skill in the art that many forms of shielding are available to a manufacturer or designer and that these forms are contemplated herein.

Optionally, the device may further comprise one or more features for preventing the inadvertent disabling of the device's vacuum pressure. For example, an embodiment of the device that comprises two plates as part of the means of generating vacuum may further comprise a feature such as a physical interference, lock, or spacer that prevents the plates from being advanced towards each other. In one example, the physical interference may be a length of material manufactured from materials known to the art including, but not limited to, aliphatic polyamides, fluorinated ethylene propylene, nylon, perfluoroalkoxy (e.g. Teflon®), polyether block amide (Pebax®), polyetheretherketone (PEEK), polyethylene, polytetrafluoroethylene (PTFE), polypropylene, polyurethane, polyvinylchloride, polysulfone, stainless steel, nickel, titanium, aluminum, brass, copper, polycarbonate, acrylic, polyoxymethylene (Delrin®), combinations and/or alloys thereof, and the like.

The length of material may be oriented generally perpendicular to the plates and positioned such that, when in place, the length of material physically and mechanically prevents the plates from being advanced towards each other. The length of material may be separate from the device (e.g., a distinct component that must be reversibly removed from the device prior to generating vacuum) or it may be joined to the device. In the latter example, the length of material may be joined to the device by a pivoting or rotatable joint or hinge. The joint or hinge may further comprise a torsion spring or other mechanism for biasing and/or maintaining the length of material in a given position and/or orientation.

For example, the length of material may be joined to the far (i.e., in a direction that is away from the housing) portion of one of the plates via a hinge and biased away from the other plate via the use of a torsion spring. The far portion of the second plate may further comprise a recess or cavity for accepting the unattached end of the length of material. Rotating the length of material about the hinge and placing the unattached end of the length of material into the recess or cavity on the second plate creates a physical or mechanical interference to prevent the plates from moving towards each other. The recess or cavity in the second plate may further comprise a mechanism for locking or holding the end of the length of material in place. The mechanism for reversibly locking or holding the end of the length of material in place may be activated by an actuator such as a button, lever, switch or the like. Alternatively, other mechanisms for preventing premature or inadvertent disablement of the vacuum pressure in the device are contemplated, including but not limited to, fixed or movable pins, locks and keys, slides, rotatable switches or toggles, combinations thereof, and the like. It should be clear to one of ordinary skill in the art that various mechanisms for restricting the activation of the features for generating pressure until a certain set of conditions or parameters is met exist and are contemplated for use in the device.

### BRIEF DESCRIPTION OF THE DRAWINGS

The invention is best understood from the following detailed description when read in conjunction with the accompanying drawings. It is emphasized that, according to common practice, the various features of the drawings are not to-scale. On the contrary, the dimensions of the various features are arbitrarily expanded or reduced for clarity. Included in the drawings are the following figures.

FIGS. 1A through 1B are schematic illustrations of the different states of flow enabled by an exemplary embodiment of the device.

FIG. 2 is a perspective view of an embodiment of the device.

FIG. 3 is a perspective view of the embodiment of the device shown in FIG. 2 illustrating the use of the extended recess to secure the proximal portion of the elongate member.

FIG. 4 is a side view of the device of FIGS. 2 and 3.

FIG. 4A is a cross-sectional view of the interaction between elements of the device taken along line A-A of FIG. 4.

FIG. 5 is a top view of the device in the configuration depicted in FIG. 3.

FIG. 6A through 6D are schematic illustrations of the different states of flow enabled by an exemplary device and system.

FIG. 7 is a schematic illustration of an embodiment of a device comprising at least one valve incorporated into the housing.

FIG. 8 is a schematic illustration of another embodiment of a device comprising at least two valves incorporated into the housing.

### DETAILED DESCRIPTION OF THE EXEMPLARY EMBODIMENTS

Before the exemplary embodiments are described, it is to be understood that this invention is not limited to particular embodiments described, as such may, of course, vary. It is also to be understood that the terminology used herein is for the purpose of describing particular embodiments only, and is not intended to be limiting, since the scope will be limited only by the appended claims.

Where a range of values is provided, it is understood that each intervening value, to the tenth of the unit of the lower limit unless the context clearly dictates otherwise, between the upper and lower limits of that range is also specifically disclosed. Each smaller range between any stated value or intervening value in a stated range and any other stated or intervening value in that stated range is encompassed within the recited range. The upper and lower limits of these smaller ranges may independently be included or excluded in the range, and each range where either, neither, or both limits are included in the smaller ranges is also encompassed within the recited range, subject to any specifically excluded limit in the stated range. Where the stated range includes one or both of the limits, ranges excluding either or both of those included limits are also included in the recited range.

Unless defined otherwise, all technical and scientific terms used herein have the same meaning as commonly understood by one of ordinary skill in the art. Although any methods and materials similar or equivalent to those described herein can be used in the practice or testing of the embodiments described, some potential and preferred methods and materials are now described. All publications mentioned herein are incorporated herein by reference to disclose and describe the methods and/or materials in connection with which the publications are cited. It is understood that the present disclosure supersedes any disclosure of an incorporated publication to the extent there is a contradiction.

It must be noted that as used herein and in the appended claims, the singular forms “a,” “an,” and “the” include plural referents unless the context clearly dictates otherwise. Thus, for example, reference to “a compound” includes a plurality of such compounds and reference to “the polymer” includes reference to one or more polymer and equivalents thereof known to those skilled in the art, and so forth.

The publications discussed herein are provided solely for their disclosure prior to the filing date of the present application. Nothing herein is to be construed as an admission that the present invention is not entitled to antedate such publication by virtue of prior invention. Further, the dates of publication provided may be different from the actual publication dates which may need to be independently confirmed.

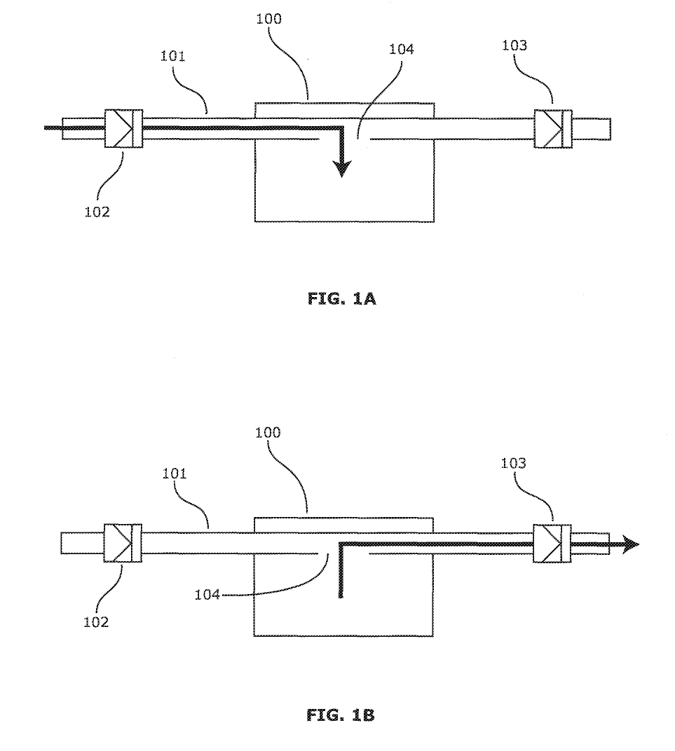

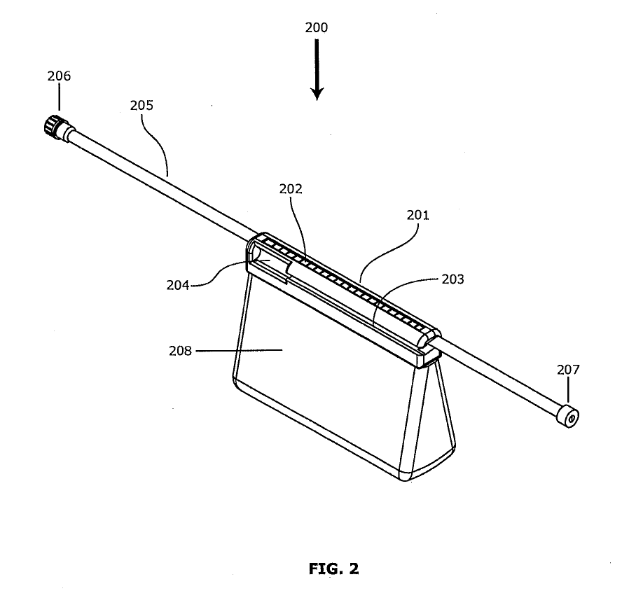

FIGS. 1A and 1B are schematic illustrations of the flow paths through the components of the device when the device is in one of two states. The device is arranged identically in each of the figures and comprises the following components: an elongate tubular member 101 containing a distal one-way valve 102, a proximal one way valve 103, and an opening 104 joined to collection reservoir 100. Distal one-way valve 102 and proximal one-way valve 103 are oriented such that flow through elongate member tubular member 101 is possible in the distal to proximal direction and prevented in the proximal to distal direction. The black arrow in FIG. 1A illustrates the direction of flow when the collection reservoir 100 is at a pressure that is below that of the external environment (i.e., a vacuum pressure within collection reservoir 100). Fluid passes through distal one-way valve 102, along elongate member 101, through opening 104 and into the collection reservoir 100. FIG. 1B illustrates the direction of flow when the collection reservoir 100 is at a pressure greater than that of the external environment. Fluid passes from the collection reservoir 100, through opening 104, along elongate member 101, through proximal one-way valve 103 and into the external environment.

FIG. 2 is a perspective view of an embodiment of the device 200, comprising an elongate member 205, a housing 201, one way valves 206 and 207, and collection reservoir 208. Elongate member 205 is disposed within and joined to housing 201. Elongate member 205 further comprises a lumen (not shown) that is in communication with the collection reservoir 208. In this embodiment, one-way valves 206 and 207 are duckbill valves and are oriented such that flow through the lumen of elongate member 205 is possible in the distal to proximal direction and prevented in the proximal to distal direction. One-way valve 206 is joined to the distal end of elongate member 205 and further comprises a male luer-lock fitting (not shown) on the distal end of one-way valve 206. One-way valve 207 is joined to the proximal end of elongate member 205.

Housing 201 further comprises an observation window 202 enabling a patient or physician to determine how much fluid exudate is resident within the collection reservoir 208, a extended recess 203, and pocket 204. Extended recess 203 may be sized smaller than the outer diameter of elongate member 205. Pocket 204 is sized to accept one-way valve 207. The external faces of the housing 201 feature relatively flat and smooth surfaces and the edges are rounded, filleted, or contoured to enhance patient comfort during use. This may be useful in cases where the device is in a body position whereby the user may be lying or sitting over the device. In this exemplary scenario, the external surface of the device that is in direct contact with the user does not present discomfort to the user such as pinching or poking of the skin or tissue which can potentially result in trauma, irritation or injury such as abrasions, cuts, bruising, ulcerations and the like.

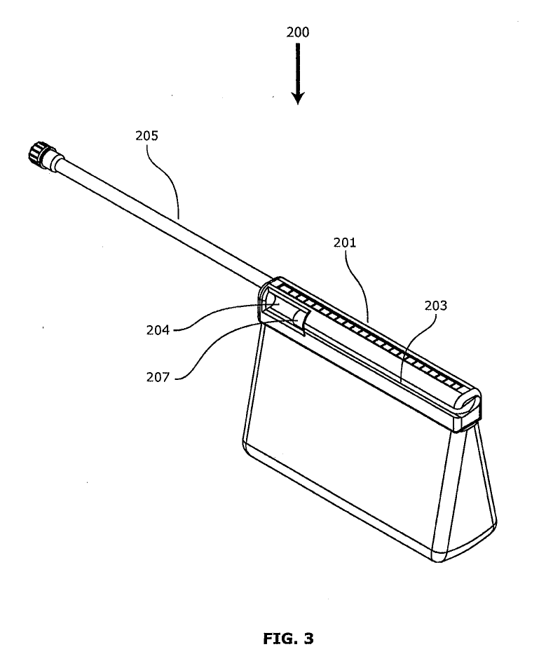

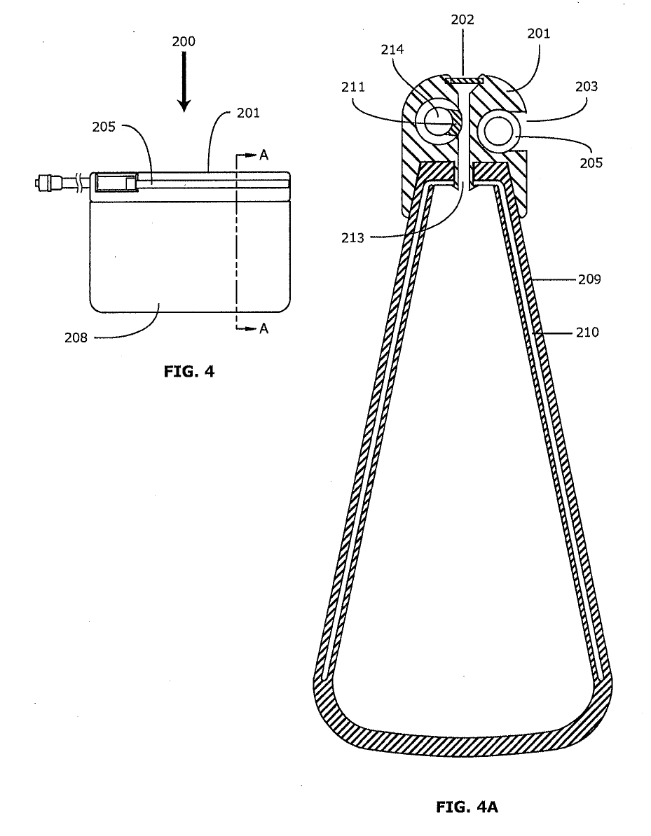

FIG. 3 is a perspective view and FIG. 4 is a side view of the embodiment 200, wherein the one-way valve 207 and the proximal portion of elongate member 205 are secured within pocket 204 and extended recess 203, respectively. In this configuration, the elongate member 205 is bent or kinked about the proximal edge of extended recess 203 within the housing 201 sufficiently to obstruct flow through its lumen in any direction thereby providing security against inadvertent loss or spillage of fluid.

FIG. 4A is a cross-sectional view of the embodiment 200 describing the relationship between the housing 201, elongate member 205, and collection reservoir 208. This view depicts additional elements of each of the major components of the device. Elongate member 205 further comprises lumen 214 and opening 211. Housing 201 further comprises port 213, observation window 202, and extended recess 203. Collection reservoir 208 further comprises membrane 209 and living hinge 210. Elongate member 205 is disposed within housing 201 such that opening 211 allows lumen 214 to communicate with port 213 in housing 201. Furthermore, the proximal portion of elongate member 205 is secured within extended recess 203 as previously described. Port 213 is sealed by observation window 202. Living hinge 210 is embedded within membrane 209 to form collection reservoir 208. In this embodiment, membrane 209 is fabricated from a soft, opaque, flexible, gas-impermeable material. Both living hinge 210 and membrane 209 are joined to housing 201 such that an air-tight seal is formed between housing 201 and collection reservoir 208. Compression of living hinge 210 decreases the internal volume of collection reservoir 208 thereby generating a negative pressure within the device which evacuates exudate from the collection reservoir 208. Evacuation of the exudate from the collection reservoir 208 in this manner requires that the proximal portion of elongate member 205 be released from extended recess 203. In doing so, the constriction at the bent or kinked portion of lumen 214 of elongate member 205 is opened to allow unrestricted passage of fluid.

FIG. 5 is a top view of the device 200, showing the arrangement of housing 201 and observation window 202. Observation window 202 is oriented parallel to the long axis of housing 201 and allows a patient, physician, or other caregiver to view the contents of collection reservoir 208. While observation window 202 is specified in FIG. 5 as a component of housing 201 and oriented parallel to the long axis of housing 201, other configurations (e.g. embedding the observation window within the membrane of the collection reservoir) have been duly contemplated. Note that proximal one-way valve 207 is secured within pocket 204. While the graduation marks adjacent to observation window 202 are illustrated in this example use cubic centimeters (i.e., cc) as the unit of measure, it should be clear to one of skill in the art that the relative sizes of the components, size of the collection reservoir 208, and the corresponding scale and choice of unit of measure of the graduation marks adjacent to the observation window 202 may be altered to accommodate any range of fluid collection applications.

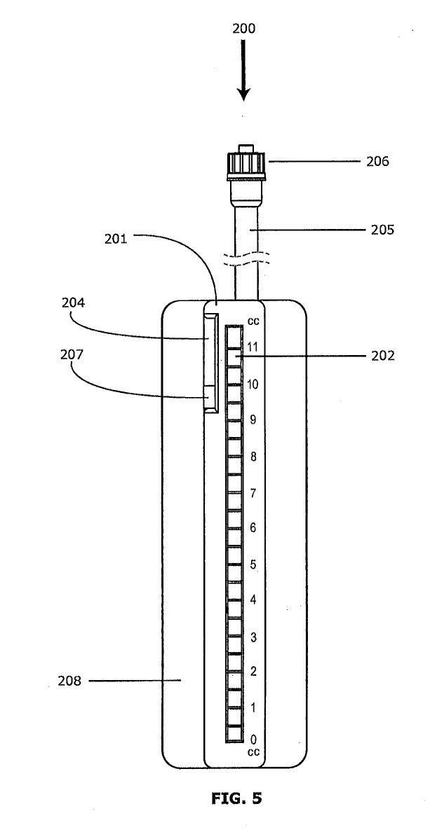

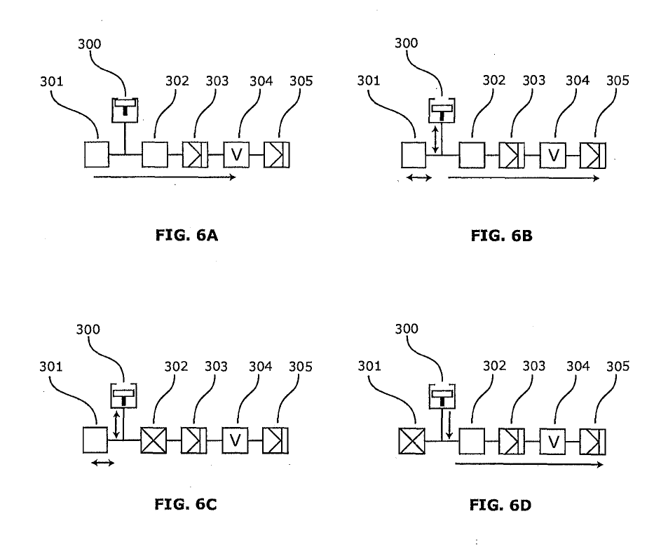

FIGS. 6A through 6D are schematic illustrations of the fluid flow paths through the internal components of the device when the device comprises elements of the valve device described in U.S. patent application Ser. No. 60/083,142. Direction of fluid flow is illustrated in FIGS. 6A to 6D using the arrows within each schematic. The elements of the valve device comprise a luer-activated valve 300 known in the art and elongate members 301 and 302 that are, in this example, incorporated into the housing (not shown) of the device. The device is arranged identically in each of the FIGS. 6A to 6D; the luer-activated valve 300 is joined to elongate members 301 and 302 such that luer-activated valve 300, elongate member 301 and elongate member 302 are in fluid communication with each other. Elongate member 302 is further connected to and in fluid communication with one-way valve 303, which is in turn connected to and in fluid communication with vacuum source 304, which is in turn connected to and in fluid communication with one-way valve 305.

FIG. 6A illustrates the device when no infusion syringe is connected to the luer-activated valve 300 and the vacuum source 304 is generating negative pressure. In this embodiment, fluid can flow between elongate member 301 and vacuum source 304 in one direction. The closure of luer-activated valve 300 and the orientation of one-way valve 305 maintain negative pressure within the system as long as vacuum source 304 is active (i.e., under negative pressure).

FIG. 6B illustrates the device once a luer-tipped coupling is inserted into the inlet of the luer-activated valve 300. Flow is possible in two directions between luer-activated valve 300 and elongate member 301 and flow is simultaneously possible in one direction between luer-activated valve 300 and the remaining components (elongate member 302, one-way valve 303, vacuum source 304 one-way valve 305) due to the presence of one-way valves 303 and 305. FIG. 6C illustrates the flow paths present in the device when a luer-tipped coupling is inserted into the inlet of the luer-activated valve 300 and elongate member 302 is compressed, blocking flow through the lumen of elongate member 302. In this state, bi-directional flow is possible between elongate member 301 and luer-activated valve 300.

FIG. 6D illustrates the flow paths present in the device when a luer-tipped coupling is inserted into the inlet of the luer-activated valve 300, elongate member 301 is compressed, and vacuum source 304 is inactive (i.e., no longer under negative pressure or neutral). The compression of elongate member 301 blocks flow through elongate member 301, enabling flow in one direction between luer-activated valve 300 and the remaining of the components (elongate member 302, one-way valve 303, vacuum source 304 one-way valve 305).

It is contemplated that all embodiments provided in FIGS. 6A to 6D may incorporate any of the design elements described in FIGS. 1 5. Specifically, the device may incorporate an elongate member 205 and an extended recess 203 such as those shown in FIG. 4, as well as an pocket 204 such as that shown in FIG. 3. This configuration would allow the elongate member 205 to be bent or kinked to provide an additional security measure against inadvertent or accidental fluid release from the device.

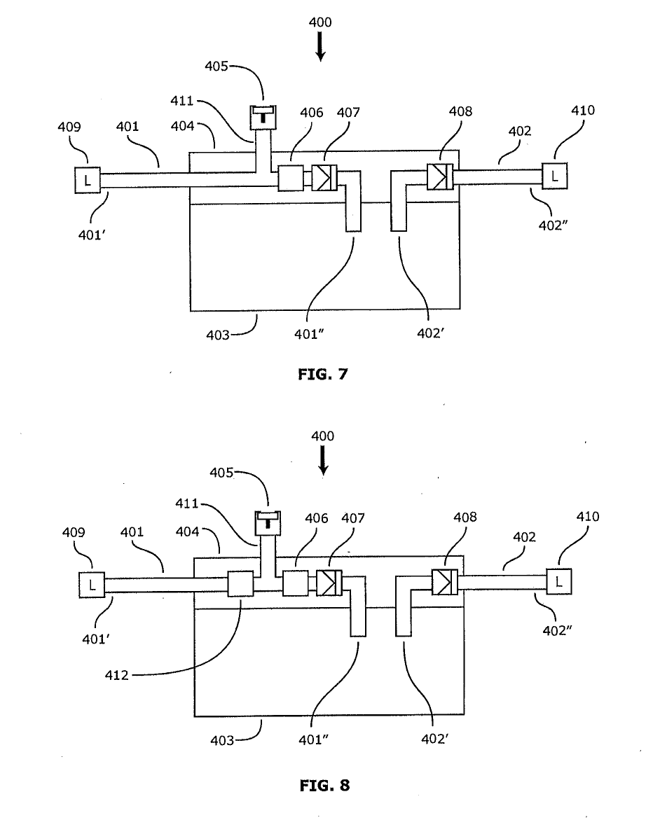

FIG. 7 is a schematic illustration of the components of another embodiment of the invention 400 comprising elements of the valve device described in U.S. patent application Ser. No. 60/083,142. The device 400 comprises a first elongate member 401, a second elongate member 402, a collection reservoir 403, a housing 404, a luer activated valve 405, a first valve 406, a first one-way valve 407, a second one-way valve 408, a first connector 409, and a second connector 410.

First elongate member 401 comprises a distal end 401′, a proximal end 401″, and at least one lumen extending therethrough. First elongate member 401 further comprises a t-connection 411 that is in fluid communication with the lumen of first elongate member 401 and terminates in luer activated valve 405. At least a portion of the length of first elongate member 401 is at least partially enclosed by housing 404 and the proximal end 401″ of first elongate member 401 is in fluid communication with the collection reservoir 403. The distal end 401′ of first elongate member 401 is joined to and in fluid communication with the first connector 409. First elongate member 401 may further comprise a first valve 406 and a first one-way valve 407, wherein the first one way valve 407 is located proximal to the first valve 406.

Second elongate member 402 comprises a distal end 402′, a proximal end 402″, and at least one lumen extending therethrough. A portion of the length of second elongate member 402 is at least partially enclosed by housing 404 and the distal end 402′ of first elongate member 401 is in fluid communication with the collection reservoir 403. The proximal end 402″ of second elongate member 402 is joined to and in fluid communication with the second connector 410. Second elongate member 402 may further comprise a one-wave valve 408. Collection volume 403 may further function as a vacuum source as previously described herein.

Likewise, housing 404 may comprise features and functions previously described herein, such as recesses, means of connection to collection reservoir 403, and the like. Luer activated valve 405 may preferably be biased into a closed position, such that in the absence of a complementary luer fitting (e.g., a mated male luer for a female luer activated valve) flow through luer activated valve 405 is prevented. The first valve 406 may be any of those described in patent application Ser. No. 60/083,142, and may be biased in the open position such that flow through the first valve 406 is allowed in the neutral or un-active state.

One-way valve 407 may be any design that at least substantially allows flow in one direction and at least substantially prevents flow in the opposite direction. For example, one-way valve 407 may any known to the art including but not limited to a check valve, duckbill valve, crack valve, ball valve, ball and spring valve, umbrella valve, flapper valve, clapper valve, diaphragm check valve, and the like. One-way valve 407 is oriented such that flow is allowed from the distal end 401′ of first elongate member 401 towards the proximal end of the proximal end 401″ first elongate member 401.

One-way valve 408 may be any design that at least substantially allows flow in one direction and at least substantially prevents flow in the opposite direction. For example, one-way valve 408 may any known to the art including but not limited to a check valve, duckbill valve, crack valve, ball valve, ball and spring valve, umbrella valve, flapper valve, clapper valve, diaphragm check valve, and the like. One-way valve 408 is oriented such that flow is allowed from the distal end 402′ of second elongate member 402 towards the proximal end of the proximal end 402″ second elongate member 402. In an exemplary embodiment, the first connector 409 may be a male luer lock and second connector 410 may be a female luer lock. Additionally (not shown), embodiment of the device 400 may further comprise impermeable caps that are respectively mated to the first and second connectors 409 and 410.

In one embodiment of the device 400, the resistance to flow in the section of first elongate member 401 lying between the luer activated valve 405 and the proximal end 401″ of first elongate member 401 may be lower than that of the portion of first elongate member 401 distal to luer activated valve 405. In this manner flow is directed from luer activated valve 405 through first valve 406, one-way valve 407, out of proximal end 401″ of first elongate member 401, and into the collection reservoir 403. A user wishing to direct flow or flush from luer activated valve 405 towards the proximal end 401′ of first elongate member 401 would need to activate valve 406. For example, the lumen of first elongate member 401 proximal to luer activated valve 405 (and the lumens of valve 406 and one-way valve 407) may be larger than the lumen of first elongate member 401 distal to luer activated valve 405.

A vacuum pressure may be generated in collection reservoir 403 by removing the optional cap from second connector 410 and compressing, squeezing, or applying positive pressure to the outer walls of collection reservoir 403. When doing so, air, exudate, effusion, exudate or other fluids/solids contained within collection reservoir 403 are prevented from flowing through first elongate member 401 by first one-way valve 407, and directed through second one way valve 408 and out of second elongate member 402 and second connector 410.

When positive pressure applied to the collection reservoir 403 is released, second one-way valve 408 prevents the ingress of air or any other external fluid back into collection reservoir 403. The optional cap may optionally be re-connected to second connector to provide a secondary means of preventing inadvertent expulsion of fluids/solids in collection reservoir 403 when the second elongate member 402 is not also pinched, kinked or closed. The second connector 410 (capped or un-capped) may be secured in an external recess of housing 404 as previously described herein. The act of securing second connector 401 in a recess of housing 404 may provide a mechanism for preventing flow through second elongate member 402 by pinching, kinking or closing second elongate member 402, e.g., as shown for embodiment 200 in FIG. 2 and FIG. 3.

While FIG. 2 and FIG. 3 are shown with a single elongate member 205, it should be clear to one of skill in the art that the concepts and designs presented in FIG. 2 and FIG. 3 may easily be extended to an embodiment of the device comprising multiple elongate members such as device 400. The negative pressure present in collection reservoir 403 may induce flow from first elongate member 401 into collection reservoir 403. This negative pressure may, for example, be used to draw exudate from a wound in a patient through an indwelling drainage line or drainage catheter in fluid communication with the first connector 409.

FIG. 8 depicts yet another embodiment of the device 400 comprising a second valve 412 located distal to the junction of t-connection 411 and first elongate member 401 and in fluid communication with first elongate member 401. The second valve 412 may be any of those described patent application Ser. No. 60/083,142, and may be biased in the open position such that flow through the second valve 412 is allowed in the neutral or un-active state. The incorporation of second valve 412 into embodiment of the device may provide an optional mechanism for preventing flow through the proximal portion of first elongate member of 401 when activated thereby enabling active flushing of proximal end 401″ of elongate member 401 using a syringe connected to luer activated valve 405.

The preceding merely illustrates the principles of the invention. It will be appreciated that those skilled in the art will be able to devise various arrangements which, although not explicitly described or shown herein, embody the principles of the invention and are included within its spirit and scope. Furthermore, all examples and conditional language recited herein are principally intended to aid the reader in understanding the principles of the invention and the concepts contributed by the inventors to furthering the art, and are to be construed as being without limitation to such specifically recited examples and conditions. Moreover, all statements herein reciting principles, aspects, and embodiments of the invention as well as specific examples thereof, are intended to encompass both structural and functional equivalents thereof Additionally, it is intended that such equivalents include both currently known equivalents and equivalents developed in the future, i.e., any elements developed that perform the same function, regardless of structure. The scope of the present invention, therefore, is not intended to be limited to the exemplary embodiments shown and described herein. Rather, the scope of present invention is embodied by the appended claims.

We claim:

1. A container for collecting fluid, comprising:
a reservoir comprising a plurality of panels enclosing an interior thereof, the panels comprising side panels generally opposite one another that are movable from an expanded position towards one another to a compressed position and biased to the expanded position for generating a vacuum within the interior;
an elongate housing extending along a top side of the reservoir and comprising a proximal end, a distal end, and a passage between the proximal and distal ends communicating with the interior of the reservoir;
an elongate member extending through the housing between the proximal and distal ends, the elongate member comprising a distal extension extending from the housing distal end, a proximal extension extending from the housing proximal end, and a lumen extending between the distal and proximal extensions, the lumen comprising an opening communicating with the passage;
a distal one-way valve coupled to the distal extension for permitting fluid flow proximally from the distal extension into the interior of the reservoir via the lumen and opening while preventing fluid flow distally out the distal extension; and
a proximal one-way valve coupled to the proximal extension for permitting fluid flow proximally from the proximal extension while preventing fluid flow distally into the proximal extension and lumen.

2. The container of claim 1, wherein the passage extends longitudinally along the housing at least partially between the proximal and distal ends, and wherein the housing further comprises a window extending between the proximal and distal ends to permit viewing fluid within the passage.

3. The container of claim 2, further comprising indicia on or adjacent the window to indicate a volume of fluid within the interior of the reservoir and passage when the housing is oriented substantially vertically.

4. The container of claim 1, further comprising an elongate recess in the housing extending partially from the proximal end towards the distal end, the recess configured to receive the proximal tubular extension when the proximal tubular extension is folded and inserted into the recess, thereby preventing fluid flow through the proximal tubular extension.

5. The container of claim 4, wherein the recess comprises (1) a first region having a cross-section smaller than the tubular extension such that the tubular extension is compressed when inserted into the recess and (2) a second region larger than the first region configured to receive the proximal one-way valve when the tubular extension is inserted into the recess.

6. The container of claim 1, further comprising a visual indicator on the housing or the reservoir configured to provide a visual output indicating pressure status of the interior of the reservoir.

7. The container of claim 1, wherein the elongate member is substantially permanently fixed relative to the housing such that a fixed length of the elongate member defines the proximal and distal extensions.

8. The container of claim 1, wherein the side panels pivot relative to one another when directed between the expanded and compressed positions.

9. The container of claim 8, wherein the side panels define planes that intersect one another in the expanded position.

10. A container for collecting fluid, comprising:
a reservoir comprising a plurality of panels enclosing an interior thereof, the panels comprising side panels generally opposite one another that are movable from an expanded position towards one another to a compressed position and biased to the expanded position for generating a vacuum within the interior;
an elongate housing comprising a valve device extending along a top side of the reservoir and comprising a proximal end, a distal end, and a passage between the proximal and distal ends communicating with the interior of the reservoir, the valve device comprising an elongate member having a proximal end, a distal end, and a lumen therethrough extending between the proximal and distal ends of the valve device, and at least two ports communicating with the elongate member;
wherein the elongate member communicates with a distal one-way valve and the passage;
wherein the distal one-way valve is coupled to the elongate member permitting fluid flow proximally from the valve device while preventing fluid flow distally;
a proximal extension having a proximal and distal end with a lumen therethrough coupled to the proximal end of the elongate housing communicating with the passage; and
a proximal one-way valve coupled to the proximal extension for permitting fluid flow proximally from the proximal extension while preventing fluid flow distally into the proximal extension and lumen.

11. The container of claim 10, wherein the valve device comprises a first port that may be selectively opened for infusion of fluids and a second port configured to couple to a catheter.

12. The container of claim 10, further comprising an actuator for selectively isolating the second port from the first port.

13. The container of claim 10, further comprising a source of fluid coupled to the first port to deliver fluid from the source via the first port into the proximal extension when the actuator isolates the second port from the first port.

14. The container of claim 10, further comprising an actuator for selectively isolating the distal one-way valve, the reservoir, and the proximal extension from the first port.

15. The container of claim 14, further comprising a source of fluid coupled to the first port to deliver fluid from the source via the first port into the second port when the actuator isolates the proximal extension from the first port.

16. The container of claim 10, further comprising a safety lever engageable to prevent the side panels from being subsequently directed towards the compressed position to maintain the vacuum within the interior.

17. A method for collecting fluid within a container, comprising:
providing a container comprising a reservoir defining an interior and compressible to from an expanded position to a compressed position to reduce a volume of the interior, the container further comprising an elongate member coupled to a housing coupled to the reservoir, the elongate member comprising a fluid path from a distal extension through the housing to a proximal extension that communicates with the interior of the reservoir;
coupling a drainage device to the distal extension;
compressing the reservoir to the compressed position, a one-way valve in the distal extension preventing air within the interior from flowing back through the distal extension into the drainage device such that the air is evacuated out the proximal extension;
releasing the reservoir such that reservoir is biased to return towards the expanded position, thereby generating a vacuum within the interior to draw fluid from the drainage device through the distal extension into the interior; and
securing the proximal extension within a recess in the housing to prevent leakage from the interior via the proximal extension.

18. The method of claim 17, wherein securing the proximal extension comprising folding the proximal extension back towards the distal extension and inserting the proximal extension into the recess such that a one-way valve on the proximal extension is received in a pocket adjacent the recess.

19. The method of claim 17, further comprising orienting the housing such that a window on the housing is oriented substantially vertically, whereby fluid within the interior flows through a passage in the housing adjacent the window to align with indicia on or adjacent the window to identify the volume of the fluid within the reservoir.

20. The method of claim 17, further comprising, after fluid has been collected within the interior:
removing the proximal extension from the recess; and
compressing the reservoir to evacuate fluid from the interior out the proximal extension, the one-way valve in the distal extension preventing the fluid from flowing back through the distal extension into the drainage device.
